(19) United States
(12) Patent Application Publication
(10) Pub. No.: US 2023/0210445 A1
(43) Pub. Date: Jul. 6, 2023

(54) Active Implantable Medical Device Capable of Distinguishing Between a Local Infection and a Systemic Infection

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 17/926,298

(22) PCT Filed: May 12, 2021
(86) PCT No.: PCT/EP2021/062607

§ 371 Date: Nov. 18, 2022

(30) Foreign Application Priority Data
May 26, 2020 (EP) 20176420.6
Sep. 15, 2020 (EP) 20196094.5

(51) Int. Cl. A61B 5/00; A61B 5/0205

(52) U.S. Cl. CPC A61B 5/412; A61B 5/02055; A61B 5/746; A61B 5/686; A61B 5/0031; A61B 5/0008; A61B 5/024

(57) ABSTRACT

An active implantable medical device (IMD) which, in operation: repeatedly senses a body temperature of a person having the IMD implanted therein; stores the sensed body temperature values; repeatedly senses a cardiac physiologic parameter of the person; stores the sensed cardiac physiologic parameter values; determines whether one of the body temperature values exceeds a predefined body temperature threshold; if an excess is determined, the IMD: determines whether one of the cardiac physiologic parameter values exceeds a predefined cardiac physiologic parameter threshold, wherein the one cardiac physiologic parameter value is determined in the same time period as the one body temperature value exceeding the body temperature threshold; classifies an infection of the person as a local infection if the one cardiac physiologic parameter value does not exceed the threshold; or classifies an infection of the person as a systemic infection if the one cardiac physiologic parameter value exceeds the threshold.

### CROSS-REFERENCE TO RELATED APPLICATIONS

This application is the United States National Phase under 35 U.S.C. § 371 of PCT International Patent Application No. PCT/EP2021/062607, filed on May 12, 2021, which claims the benefit of European Patent Application No. 20196094.5, filed on Sep. 15, 2020, and European Patent Application No. 20176420.6, filed on May 26, 2020, the disclosures of which are hereby incorporated by reference herein in their entireties.

### TECHNICAL FIELD

The present invention relates to an active implantable medical device according to the preamble of claim 1, to an arrangement comprising such an active implantable medical device according to the preamble of claim 9, and to a computer program product according to the preamble of claim 12.

### BACKGROUND

An increase in body temperature is an early indicator for infection, e.g., for a viral infection such as influenza or COVID-19. In case of COVID-19, the available clinical data shows that patients with heart diseases appear to have a higher mortality rate compared to patients without such diseases. An increase in body temperature can, however, also be an indicator of a local infection, such as a local pocket infection.

In case of a bigger outbreak of infection, in particular of a pandemic, such as the COVID-19 pandemic, it is important to take the necessary clinic and pandemic measures in dependence on the type of detected infection. If a patient suffers only from a local pocket infection, it is neither necessary nor advisable to further examine or treat the patient in a COVID-19 center. However, if there is a reasonable suspicion of a COVID-19 infection in case of a systemic infection, it is relevant to further examine and treat the patient in a COVID-19 center and to subject the patient to the established clinical and pandemic workflows.

Active implantable medical devices such as active cardiac implants are capable of collecting an implant temperature which directly corresponds to the body temperature of a person to whom the device has been implanted.

The present disclosure is directed toward overcoming one or more of the above-mentioned problems, though not necessarily limited to embodiments that do.

### SUMMARY

It is an object of the present invention to provide a novel possibility of distinguishing between a local infection and a systemic infection of a patient carrying an active implantable medical device in an implanted state.

At least this object is achieved with an active implantable medical device having the claim elements of claim 1. Such a device comprises a processor, a memory unit, a detection unit and a temperature sensor. The detection unit serves for detecting a cardiac physiologic parameter. The temperature sensor serves for sensing a body temperature.

According to an aspect of the presently claimed invention, the memory unit comprises a computer-readable program that causes the processor to perform the steps explained the following when executed on the processor.

First, the temperature sensor is caused to sense the body temperature of the person to whom the active implantable medical device is implanted. In this context, the temperature sensor is caused to sense the body temperature a plurality of times so that a plurality of time-dependent body temperature values is obtained. The obtained body temperature values are stored in the memory unit.

Furthermore, the detection unit is caused to sense a cardiac physiologic parameter of the same person. In this context, the detection unit is caused to sense the cardiac physiologic parameter also a plurality of times so that a plurality of time-dependent cardiac physiologic parameter values is obtained. The obtained cardiac physiologic parameter values are then stored in the memory unit.

In another step, it is determined whether at least one of the body temperature values stored in the memory unit exceeds a predefined body temperature threshold. Such an excess of the predefined body temperature threshold is an indicator of infection. However, it is unclear when considering only the body temperature if the infection is a local infection or a systemic infection.

Thus, if an excess of the predefined body temperature threshold has been determined, a discrimination between a systemic infection and a local infection is carried out. For this purpose, it is determined whether at least one of the cardiac physiologic parameter values exceeds a predefined cardiac physiologic parameter threshold. In this context, the at least one of the cardiac physiologic parameter values has been determined in the same time period as the at least one of the body temperature values that exceeds the body temperature threshold. Thus, the method performed by the active implantable medical device does not only evaluate the body temperature in a specific time period or at a specific time point, but also an additional parameter, namely, a cardiac physiologic parameter that has been obtained in the same time period or at the same time point.

If the at least one of the body temperature values exceeds the body temperature threshold but if, at the same time, the at least one of the cardiac physiologic parameter values does not exceed the cardiac physiologic parameter threshold, this is taken as an indication that the person or patient suffers from a local infection. Therefore, an infection of the person (identified by the increased body temperature) is classified as a local infection.

If, however, the at least one of the body temperature values exceeds the body temperature threshold and if, at the same time, the at least one of the cardiac physiologic parameter values exceeds the cardiac physiologic parameter threshold, this is taken as an indication that the person or patient does not only suffer from a local infection, but rather from a systemic infection. Therefore, an infection of the person (identified by the increased body temperature) is classified as systemic infection.

If the infection has been classified as local infection, an advice can be given to the patient to be further examined and/or treated in the hospital or medical center that has performed the implantation of the active implantable medical device. If the infection has been classified as systemic infection, an advice can be given to the patient to contact a hospital or medical center specialized on the management and treatment of pandemic diseases. Such an advice can be specifically given if there is another pointer that the patient might suffer from a pandemic disease, e.g., a contact to an already infected person, a stay in a risk region and/or other disease-specific symptoms.

In an embodiment, the time period during which the body temperature and the cardiac physiologic parameter is determined is a time period of 6 hours to 7 days, in particular 12 hours to 6 days, in particular 18 hours to 5 days, in particular 1 day to 4 days, in particular 2 days to 3 days. It is possible that the body temperature and/or the cardiac physiologic parameter represents an average value of individual body temperature values or cardiac physiologic parameter values, respectively, determined in such a time period. Thus, the body temperature value can represent a mean body temperature calculated as an average from a plurality (two or more, e.g., 2, 3, 4, 5, 6, 7, 8, 9, 10) body temperature values measured on a single day or measured on consecutive days. Thus, the body temperature value can represent, in an embodiment, an average body temperature of a single day, of two consecutive days, or of three consecutive days. Likewise, the cardiac physiologic parameter value can represent a mean cardiac physiologic parameter calculated as an average from a plurality (two or more, e.g., 2, 3, 4, 5, 6, 7, 8, 9, 10) cardiac physiologic parameter values measured on a single day or measured on consecutive days. Thus, the cardiac physiologic parameter value can represent, in an embodiment, an average cardiac physiologic parameter value of a single day, of two consecutive days, or of three consecutive days.

In an embodiment, it is necessary that at least two (or exactly two), at least three (or exactly three), at least four (or exactly four), or at least five (or exactly five) body temperature values exceed the body temperature threshold and/or that at least two (or exactly two), at least three (or exactly three), at least four (or exactly four), or at least five (or exactly five) cardiac physiologic parameter values do not exceed the cardiac physiologic parameter threshold to classify the infection as local infection.

In an embodiment, it is necessary that at least two (or exactly two), at least three (or exactly three), at least four (or exactly four), or at least five (or exactly five) body temperature values exceed the body temperature threshold and/or that at least two (or exactly two), at least three (or exactly three), at least four (or exactly four), or at least five (or exactly five) cardiac physiologic parameter values exceed the cardiac physiologic parameter threshold to classify the infection as systemic infection.

The term “active implantable medical device” is well known to a person skilled in the art. An “active medical device” is typically defined to be any medical device relying for its functioning on a source of electrical energy or any source of power other than that directly generated by the human body or gravity.

An “active implantable medical device” is typically defined to be any active medical device which is intended to be totally or partially introduced, surgically or medically, into the human body or by medical intervention into a natural orifice, and which is intended to remain after the procedure.

Further details on active implantable medical implants can be found, e.g., in the consolidated text of the Council Directive 90/385/EEC of 20 Jun. 1990 on the approximation of the laws of the Member States relating to active implantable medical devices with subsequent amendments in the version published on 11 Oct. 2007. The consolidated text of this Council Directive is freely accessible under the following link: https://eur-lex.europa.eu/legal-content/EN/TXT/?uri=CELEX: 01990L0385-20071011.

In an embodiment, the active implantable medical device is an implantable pulse generator (IPG), an implantable cardioverter-defibrillator (ICD), a device for cardiac resynchronization therapy (CRT), a neurostimulator or an implantable cardiac monitor. Generally, the active implantable medical device can be any implantable medical device that is able to detect a cardiac physiologic parameter and a body temperature of a person, wherein it is specifically equipped to carry out the precedingly explained method.

In an embodiment, the cardiac physiologic parameter is a heart rate, a heart rate at rest, a respiratory rate, a respiratory rate at rest, a cardiac impedance, a cardiac contractility, a cardiac output, a heart rate variability, a respiratory arrhythmia, an oxygen saturation, a blood flow velocity, and/or heart sounds. In this context, the heart rate or the respiratory rate (whether or not be measured at rest) is a mean (average) rate. The mean or average can be calculated from a plurality of measurements made on one and the same day. Then, the mean average value corresponds to an average daily rate. In an embodiment, the mean average is calculated from a plurality of values obtained on different days. In an embodiment, the mean average rate is obtained from values obtained at different days but at the same time point on each day. A time point is considered to be the same time point is another time point if it does not differ from the other the time point by more than 1 second to 10 minutes, in particular 10 seconds to 9 minutes, in particular 20 seconds to 8 minutes, in particular 30 seconds to 7 minutes, in particular 40 seconds to 6 minutes, in particular 50 seconds to 5 minutes, in particular 1 minute to 4 minutes, in particular 2 minutes to 3 minutes.

In an embodiment, the cardiac physiologic parameter is a heart rate or heart with at rest, in particular an average heart rate or an average heart rate at rest. In this context, the cardiac physiologic parameter threshold is set to be a heart rate lying in a range of 80 to 110 beats per minute, in particular of 85 to 105 beats per minute, in particular of 89 to 104 beats per minute, in particular of 94 to 99 beats per minute. Then, a heart rate of, e.g., at least 81 beats per minute, in particular at least 90 beats per minute, in particular at least 95 beats per minute, in particular at least 100 beats per minute, in particular at least 105 beats per minute, in particular at least 111 beats per minute would exceed this threshold and would thus be indicative of a systemic infection if, at the same time, an increased body temperature has been detected. Expressed in other words, a heart rate or a heart rate at rest of at least 80 beats per minute, in particular at least 85 per minute, in particular at least 90 beats per minute, in particular at least 95 beats per minute, in particular at least 100 beats per minute, in particular at least 105 beats per minute is considered as an indicator of a systemic infection if an increased body temperature is detected at the same time. If the heart rate is considered as cardiac physiologic parameter, the threshold is, in an embodiment, not lower than 89 beats per minute. If the heart rate at rest is considered as cardiac physiologic parameter, the threshold is, in an embodiment, lying in a range of 80 to 89 beats per minute.

In an embodiment, the cardiac physiologic parameter is a heart rate or a heart at rest. Furthermore, the computer-readable program causes the processor to determine an excess of the cardiac physiologic parameter threshold if the heart rate or the heart rate at rest is at least 110% of the mean heart rate or the mean heart rate at rest, respectively, of a first is number of last days prior to the determining of the excess. In this context, the first number is a number chosen from a range between 3 and 400, in particular from 5 to 365, in particular from 10 to 350, in particular from 15 to 300, in particular from 20 to 250, in particular from 25 to 200, in particular from 30 to 190, in particular from 40 to 180, in particular from 50 to 150, in particular from 60 to 120, in particular from 70 to 100, in particular from 80 to 90.

In an embodiment, the body temperature threshold is a temperature lying in a range from 37.5° C. to 38.5° C., in particular from 37.6° C. to 38.4° C., in particular from 37.7° C. to 38.3° C., in particular from 37.8° C. to 38.2° C., in particular from 37.9° C. to 38.1° C. To give an example, according to the definition by the Centers for Disease Control and Prevention (CDC), a person is considered to have fever when he or she has a measured temperature of at least 38.0° C. Thus, if the body temperature threshold is set to be not higher than 37.9° C., any person having fever according to the CDC definition will be correctly identified when applying the method carried out by the presently claimed active implantable medical device.

In an embodiment, the computer-readable program causes the processor to sense the body temperature a plurality of times per day. E.g., the body temperature can be sensed 2 to 12 times a day, in particular 3 to 11 times a day, in particular 4 to 10 times a day, in particular 5 to 9 times a day, in particular 6 to 8 times a day. In an embodiment, the body temperature is sensed every hour, i.e., 24 times a day. In an embodiment, the temperature is sensed every 30 minutes, i.e., 48 times a day. In an embodiment, the body temperature is sensed in an interval lying between every 15 minutes and every 2 hours, in particular between every 30 minutes and every 1.5 hours, in particular between every 45 minutes and every hour.

In an embodiment, the computer-readable program causes the processor to sense the body temperature for a plurality of days, in particular 2 to 14 days, in particular 3 to 13 days, in particular 4 to 12 days, in particular 5 to 11 days, in particular 6 to 10 days, in particular 7 to 9 days.

In an embodiment, the computer-readable program causes the processor to output an alert if the actual body temperature value exceeds the body temperature threshold and if optionally also the cardiac physiologic parameter value exceeds the cardiac physiologic parameter threshold.

Such an alert can be used as indication of a local infection or a systemic infection of the patient carrying the implantable medical device in an implanted state is given. Medical staff, in particular a medical doctor, evaluating the patient's data can then inform the patient that he or she might be infected and can take appropriate countermeasures. Alternatively, the alert on a suspected systemic infection (e.g., a potential influenza infection or a potential COVID-19 infection) can be automatically processed and communicated to the patient.

In an aspect, the present invention relates to an arrangement comprising an active implantable medical device, in particular an active implantable medical device according to the preceding explanations, and an evaluation unit. The evaluation unit is located separate from the active implantable medical device. The active implantable medical device comprises a first processor, a first memory unit, a detection unit, a temperature sensor, and a data communication unit. The detection unit serves for detecting a cardiac physiologic parameter of the patient to whom the active implantable medical device is implanted. Likewise, the temperature sensor serves for sensing a body temperature of the same patient.

The first memory unit comprises a first computer-readable program that causes the first processor to perform the steps explained in the following when executed on the processor.

First, the processor causes the temperature sensor to repeatedly sense a body temperature of a person to whom the active implantable medical device is implanted. Thus, a plurality of body temperature values is obtained.

Furthermore, the first processor causes the detection unit to repeatedly sense a cardiac physiologic parameter of the person. Thus, a plurality of cardiac physiologic parameters of the person is obtained.

The body temperature values and the cardiac physiologic parameter values are then transmitted via the data communication unit to the evaluation unit located outside a body of the person.

The evaluation unit comprises a second processor and a second memory unit. The second memory unit comprises a second computer-readable program that causes the steps explained in the following when executed on the second processor.

First, the transmitted body temperature values and the transmitted cardiac physiologic parameter values are stored in the second memory unit.

Furthermore, it is determined whether at least one of the body temperature values exceeds a predefined body temperature threshold.

If such an excess of the predefined body temperature threshold has been determined, it is furthermore determined whether at least one of the cardiac physiologic parameter values exceeds a predefined cardiac physiologic parameter threshold. In this context, the at least one of the cardiac physiologic parameter values has been determined in the same time period or at the same time point as the at least one of the body temperature values exceeding the body temperature value threshold.

If an excess of the predefined body temperature threshold has been determined, a discrimination between a systemic infection and a local infection is carried out. For this purpose, it is determined whether at least one of the cardiac physiologic parameter values exceeds a predefined cardiac physiologic parameter threshold. In this context, the at least one of the cardiac physiologic parameter values has been determined in the same time period as the at least one of the body temperature values that exceeds the body temperature threshold. Thus, the method performed by the arrangement does not only evaluate the body temperature in a specific time period or at a specific time point, but also an additional parameter, namely, a cardiac physiologic parameter that has been obtained in the same time period or at the same time point.

If the at least one of the body temperature values exceeds the body temperature threshold but if, at the same time, the at least one of the cardiac physiologic parameter values does not exceed the cardiac physiologic parameter threshold, this is taken as an indication that the person or patient suffers from a local infection. Therefore, an infection of the person is classified as a local infection.

If, however, the at least one of the body temperature values exceeds the body temperature threshold and if, at the same time, the at least one of the cardiac physiologic parameter values exceeds the cardiac physiologic parameter threshold, this is taken as an indication that the person or patient does not only suffer from a local infection, but rather from a systemic infection. Therefore, an infection of the person is classified as systemic infection.

Thus, the arrangement serves for the same purpose as the active implantable medical device described above. However, the data evaluation and further use of the evaluated data is not directly performed within the active implantable medical device, but rather outside in an external evaluation unit. This approach requires less energy consumption of the active implantable medical device than evaluating the data within the active implantable medical device. Since the energy resources of an active implantable medical device are very limited (in particular in case of miniaturized implantable medical devices), it is generally favorable to perform energy-consuming calculation tasks outside the active implantable medical device. In such a case, additional energy is necessary for the data transmission. The data transmission requires, however, typically much less energy than calculations and data manipulation within the implantable medical device.

All embodiments explained above with respect to the memory unit and/or the processor can be transferred to the first memory unit or the first processor, respectively, and/or the second memory unit or the second processor, respectively, of the described arrangement.

In an embodiment, the data communication unit serves for transferring data to the evaluation unit in a wireless manner. All standard data transmission protocols or specifications are appropriate for such a wireless data communication. Examples of standard data transmission protocols or specifications are the Medical Device Radiocommunications Service (MICS), the Bluetooth Low Energy (BLE) protocol and the Zigbee specification.

In an embodiment, the first computer-readable program causes the first processor to transmit the body temperature values at least once a day, in particular exactly once a day, in particular 2 to 12 times a day, in particular 3 to 11 times a day, in particular 4 to 10 times a day, in particular 5 to 9 times a day, in particular 6 to 8 times a day. In an embodiment, the data is transmitted every hour, i.e., 24 times a day. In an embodiment, the data is transmitted every 30 minutes, i.e., 48 times a day. In an embodiment, the data is transmitted in an interval lying between every 15 minutes and every 2 hours, in particular between every 30 minutes and every 1.5 hours, in particular between every 45 minutes and every hour. In an embodiment, each body temperature value is transmitted directly after sensing it.

In an embodiment, the longevity of the active implantable medical device is shortened by a maximum of 10%, in particular 5%, in particular 1% when carrying out a daily data transfer with respect to an active implantable medical device performing a quarterly data transmission.

In an embodiment, the evaluation unit is located remote from the active implantable medical device. To give an example, the evaluation unit can be located within a medical center and can be arranged to receive body temperature data and other medical data such as cardiac physiologic parameter data from a plurality of active implantable medical devices. In such a case, the data communication unit of the active implantable medical device typically transfers the body temperature values to an external local data communication unit which then serves for further data transmission to the evaluation unit.

In an aspect, the present invention relates to a computer program product comprising computer-readable code that causes a processor to perform the steps explained in the following when executed on the processor.

In a first step, body temperature values obtained from a temperature sensor of an active implantable medical device and cardiac physiologic parameter values obtained from a detection unit of the same active implantable medical device are stored in a memory unit of the implantable medical device or in a memory unit of an evaluation unit located outside a body of a person to whom the active implantable medical device has been implanted.

Based on the stored body temperature values, it is determined whether at least one of the body temperature values exceeds a predefined body temperature threshold. Such an excess of the predefined body temperature threshold is an indicator of infection. However, it is unclear when considering only the body temperature if the infection is a local infection or a systemic infection.

Thus, if an excess of the predefined body temperature threshold has been determined, a discrimination between a systemic infection and a local infection is carried out. For this purpose, it is determined whether at least one of the cardiac physiologic parameter values exceeds a predefined cardiac physiologic parameter threshold. In this context, the at least one of the cardiac physiologic parameter values has been determined in the same time period as the at least one of the body temperature values that exceeds the body temperature threshold. Thus, the method caused to be performed by the computer-readable code does not only evaluate the body temperature in a specific time period or at a specific time point, but also an additional parameter, namely, a cardiac physiologic parameter that has been obtained in the same time period or at the same time point.

If the at least one of the body temperature values exceeds the body temperature threshold but if, at the same time, the at least one of the cardiac physiologic parameter values does not exceed the cardiac physiologic parameter threshold, this is taken as an indication that the person or patient suffers from a local infection. Therefore, an infection of the person is classified as a local infection.

If, however, the at least one of the body temperature values exceeds the body temperature threshold and if, at the same time, the at least one of the cardiac physiologic parameter is values exceeds the cardiac physiologic parameter threshold, this is taken as an indication that the person or patient does not only suffer from a local infection, but rather from a systemic infection. Therefore, an infection of the person is classified as systemic infection.

In an aspect, the present invention relates to a method for distinguishing between a local infection and a systemic infection of a patient to whom an active implantable medical device has been implanted. Thus, this method serves for a differential diagnosis. In this context, the method comprises the steps explained in the following.

First, the body temperature of a patient to whom an active implantable medical device has been implanted is repeatedly sensed by a temperature sensor of the active implantable medical device. The active implantable medical device further comprises a processor, a memory unit, and a detection unit configured to detect a cardiac physiologic parameter. The body temperature is sensed a plurality of times so that a plurality of time-dependent body temperature values is obtained. The obtained body temperature values are stored in the memory unit.

Furthermore, a cardiac physiologic parameter of the same patients is repeatedly sensed with the detection unit. In this context, the cardiac physiologic parameter is sensed a plurality of times so that a plurality of time-dependent cardiac physiologic parameter values is obtained. The obtained cardiac physiologic parameter values are then stored in the memory unit.

In another step, it is determined whether at least one of the body temperature values stored in the memory unit exceeds a predefined body temperature threshold. Such an excess of the predefined body temperature threshold is an indicator of infection. However, it is unclear when considering only the body temperature if the infection is a local infection or a systemic infection.

Thus, if an excess of the predefined body temperature threshold has been determined, a discrimination between a systemic infection and a local infection is carried out. For this purpose, it is determined whether at least one of the cardiac physiologic parameter values exceeds a predefined cardiac physiologic parameter threshold. In this context, the at least one of the cardiac physiologic parameter values has been determined in the same time period as the at least one of the body temperature values that exceeds the body temperature threshold. Thus, this method does not only evaluate the body temperature in a specific time period or at a specific time point, but also an additional parameter, namely, a cardiac physiologic parameter that has been obtained in the same time period or at the same time point.

If the at least one of the body temperature values exceeds the body temperature threshold but if, at the same time, the at least one of the cardiac physiologic parameter values does not exceed the cardiac physiologic parameter threshold, this is taken as an indication that the patient or patient suffers from a local infection. Therefore, an infection of the patient is classified as a local infection.

If, however, the at least one of the body temperature values exceeds the body temperature threshold and if, at the same time, the at least one of the cardiac physiologic parameter values exceeds the cardiac physiologic parameter threshold, this is taken as an indication that the patient or patient does not only suffer from a local infection, but rather from a systemic infection. Therefore, an infection of the patient is classified as systemic infection.

In an embodiment, the method further comprises a decision step in which it is decided is to examine and/or treat the patient in a hospital specialized on the treatment of infectious diseases. This decision is made if the infection of the patient has been classified as systemic infection. If, on the other hand, the infection of the patient has been classified as local infection, such as a local pocket infection, it is rather decided not to examine and/or treat the patient in a hospital specialized on the treatment of infectious diseases. Rather, the patient is then to be further examined and/or treated in the hospital that has been implanted the active implantable medical device or a hospital being likewise appropriate for an examination and/or treatment of such a local infection.

In an embodiment, the hospital is a hospital specialized on the treatment of COVID-19. In such a case, the specific clinical and pandemic workflows (such as quarantine measures) can be applied to the patient who is suspected of having a systemic infection like a viral infection such as an influenza infection or COVID-19 infection.

All embodiments of the described active implantable medical device can be combined in any desired way and can be transferred either individually or in any arbitrary combination to the described arrangement, the described computer program product and the described method. Furthermore, all embodiments of the described arrangement can be combined in any desired way and can be transferred either individually or in any arbitrary combination to the active implantable medical device, to the computer program product and to the described method. Likewise, all embodiments of the computer program product can be combined in any desired way and can be transferred either individually or in any arbitrary combination to the active implantable medical device, to the arrangement and to the described method. Lastly, all embodiments of the described method can be combined in any desired way and can be transferred either individually or in any arbitrary combination to the active implantable medical device, to the arrangement and to the computer program product.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained in the following with respect to exemplary embodiments and accompanying Figures. In the Figures:

FIG. 1A shows a plot on the mean daily body temperature of a first patient to whom an active medical implant has been implanted;

FIG. 1B shows activity data of the same patient as in FIG. 1A in the same time period;

FIG. 1C shows mean heart rate values of the same patient as in FIG. 1A in the same time period;

FIG. 1D shows mean resting heart rate values of the same patient as in FIG. 1A in the same period;

FIG. 2A shows a plot on the mean daily body temperature of a second patient to whom an active medical implant has been implanted;

FIG. 2B shows activity data of the same patient as in FIG. 2A in the same time period;

FIG. 2C shows mean heart rate values of the same patient as in FIG. 2A in the same time period;

FIG. 2D shows mean resting heart rate values of the same patient as in FIG. 2A in the same period; and

FIG. 3 shows a schematic representation of an implantable medical device of an embodiment of the present invention; and

FIG. 4 shows a schematic representation of an evaluation unit used in conjunction with the implantable medical device in accordance with an embodiment of the present invention.

### DETAILED DESCRIPTION

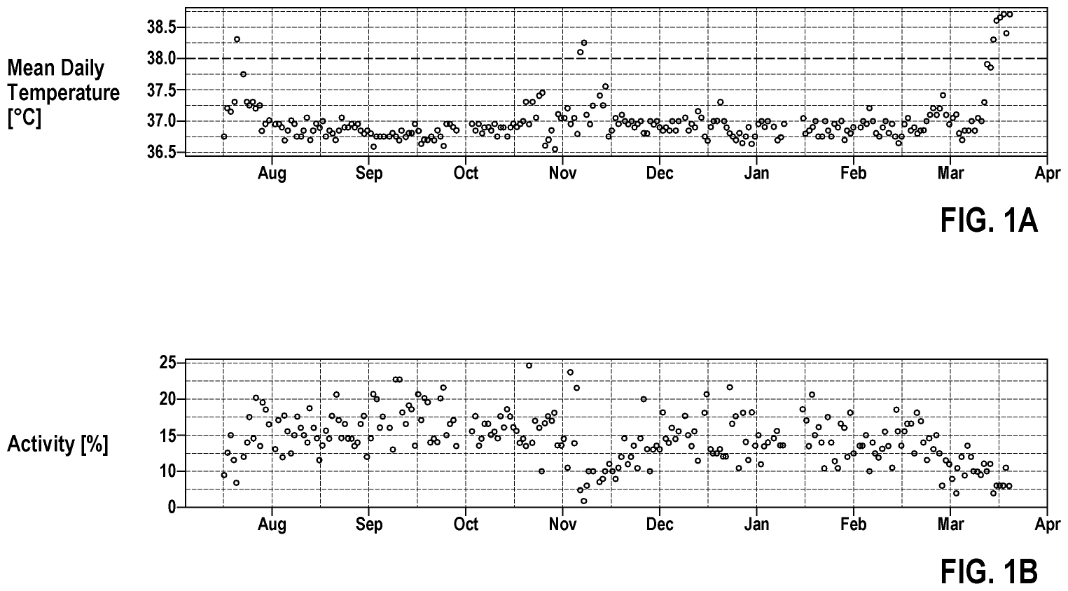

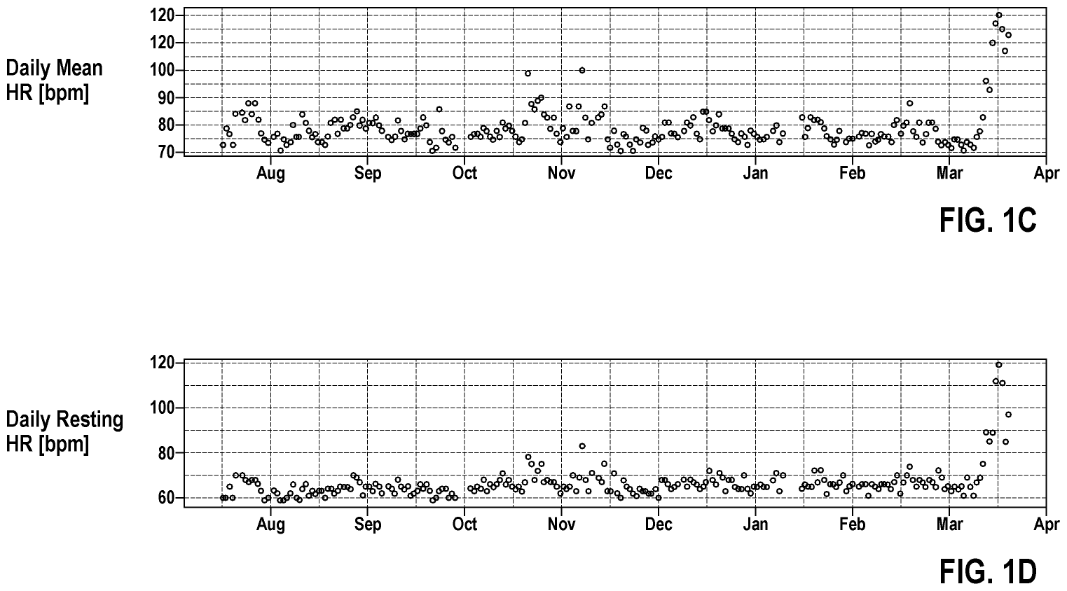

FIG. 1A shows the mean daily body temperature of a first patient carrying an active implantable medical device 10, namely, an implantable pulse generator. The body temperature is measured a plurality of times a day, wherein the individual body temperature values are averaged to obtain a mean daily body temperature. This mean daily body temperature is stored in a memory unit of the implantable pulse generator.

On a few occasions in July and November, the mean daily body temperature was equal to or higher than 38° C., i.e., it has exceeded a body temperature threshold of 37.9° C. Since the body temperature fell quickly again below the before-mentioned threshold, these events were not further evaluated in detail. However, the mean daily body temperature values of March show a higher number of individual body temperature values exceeding the body temperature threshold of 37.9° C., thus indicating an infection of the patient.

FIG. 1B shows the activity of the patient. No clear trend is observable, even though it appears that the activity in March is slightly lower than in the preceding months, in particular with respect to the last recorded March days in which the body temperature exceeded the body temperature threshold. However, the activity of the patient does not appear to be a good indicator for distinguishing a local infection from a systemic infection.

The situation is different when considering the daily mean heart rate that is plotted in FIG. 1C. It is striking that the daily mean heart rate raised in March to values significantly above 90 beats per minute (bpm). Similar daily mean heart rate values could have been observed on two single days in October and in November, wherein the November value coincided with an increased body temperature of the patient. However, this were single-day events not being indicative for a manifested infection of the patient. In contrast, in March, the daily mean heart rate was on eight days higher than 90 beats per minute.

A similar situation can be observed when considering the daily mean heart rate at rest, as can be seen from FIG. 1D. This daily mean heart rate at rest was higher than 80 beats per minute on eight days in March, but only on one day in the preceding months. Thus, the patient experienced both a body temperature of equal to or higher than 38° C. and a daily mean heart rate of higher than 90 bpm as well as a daily mean heart rate at rest of higher than 80 beats per minute on the same days in March.

This is taken as an indication of a systemic infection since both the body temperature and the further cardiac physiologic parameter have exceeded the respective threshold.

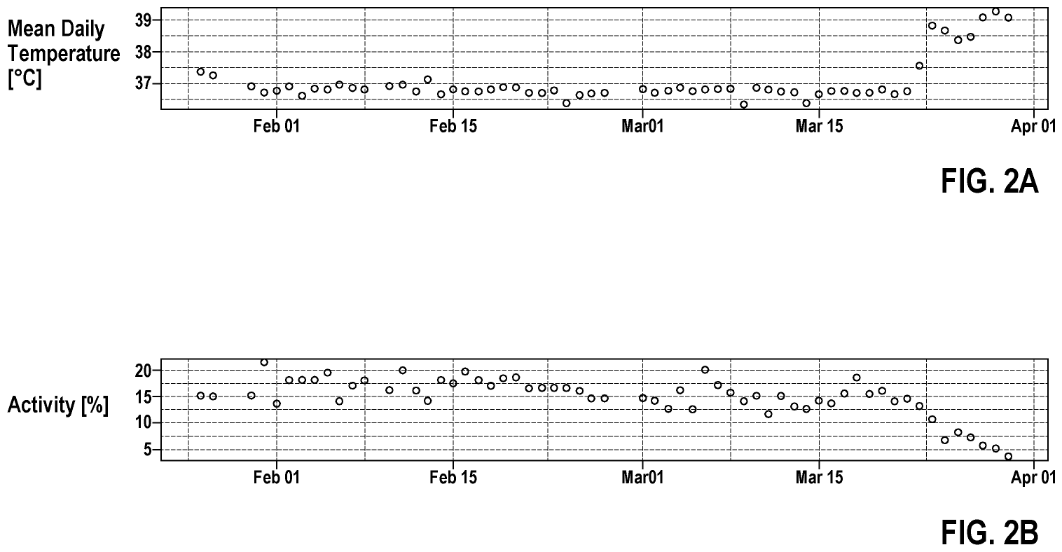

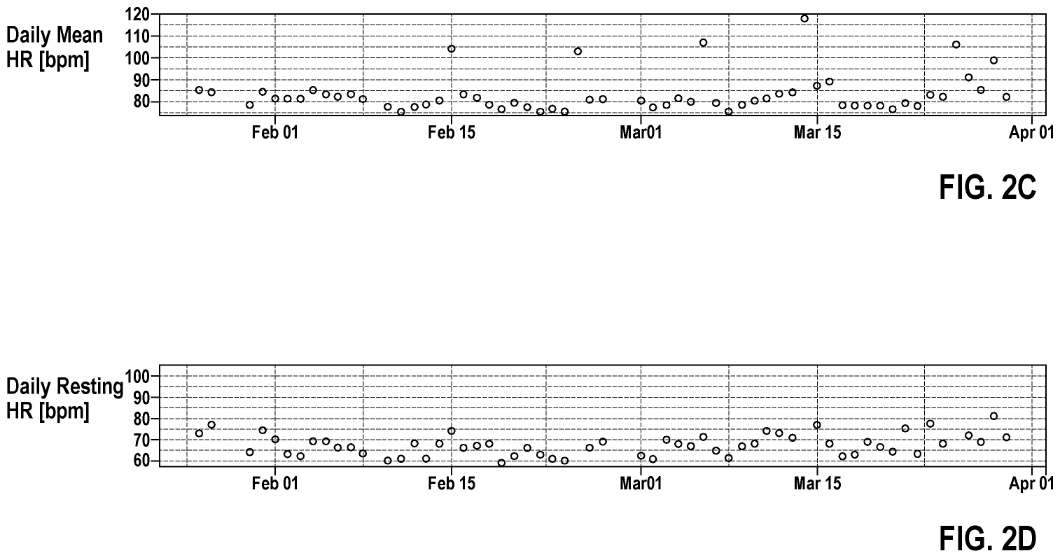

FIG. 2A shows the body temperature values of another patient also carrying an active implantable medical device 10, namely, an implantable pulse generator. Here, also a body temperature increase above 38° C. was observed in March (FIG. 2A).

Furthermore, the activity of the patient was decreased with respect to the previous days (FIG. 2B).

However, the daily mean heart rate did not significantly deviate from the previously obtained values (FIG. 2C). In particular, it did not exceed a threshold of 89 bpm over more than 2 consecutive days.

Furthermore, also the daily mean heart rate at rest did not significantly deviate from the previously obtained value and was only on one single day higher than 80 beats per minute (FIG. 2D).

When taking the body temperature values of the second patient and either the daily mean heart rate values or the daily mean heart rate values at rest of the second patient together, no clear trend of the body temperature value and at least one of the further cardiac physiologic parameters could be observed. Thus, this clearly indicates that the second patient only suffers from a local infection so that the patient is not advised to be further examined and/or treated in a hospital specialized on infectious diseases like COVID-19. Rather, the hospital or doctor who has implanted the active implantable medical device 10 should take care of the patient and treat the local (pocket) infection in a classic way as needed by the patient.

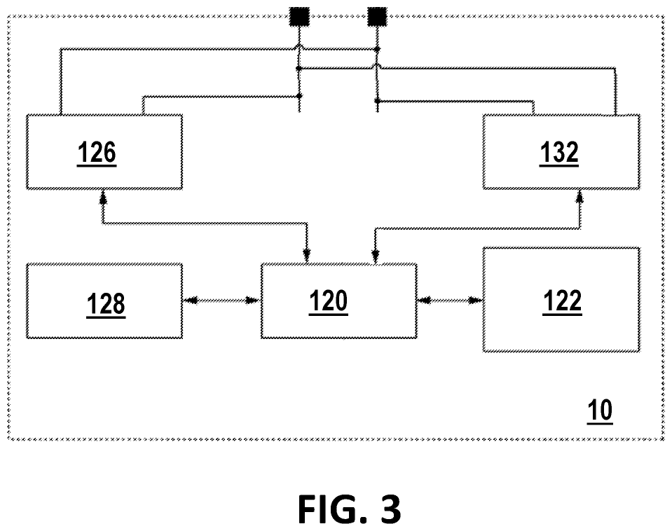

FIG. 3 shows a schematic representation of an implantable medical device 10 (e.g., an active cardiac implant) for detecting a cardiac physiologic parameter and a body temperature of a person. The active medical implant 10 includes a (first) processor 120 in two-way communication with a (first) memory unit 122, a detection unit 126, a data communication unit 128 and a temperature sensor 132. The detection unit 126 serves for detecting a cardiac physiologic parameter(s) of a patient to whom the implantable medical device 10 is implanted. The temperature sensor 132 serves for sensing a body temperature(s) of the same patient. The (first) memory unit 122 comprises a (first) computer-readable program that causes the (first) processor 120 to perform the various steps described/explained above when executed on the (first) processor 120.

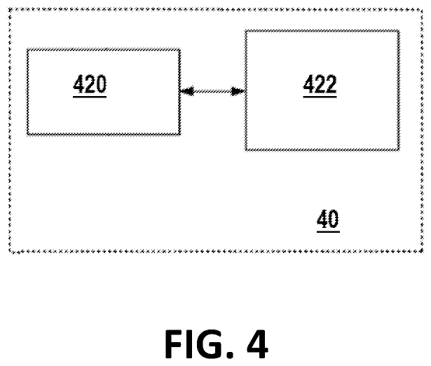

FIG. 4 shows a schematic representation of an evaluation unit 40 used in conjunction with the implantable medical device 10 in accordance with an embodiment of the present invention. The evaluation unit 40 is located separate from the active implantable medical device 10. The body temperature values measured by the temperature sensor 132 and the cardiac physiologic parameter values measured by the detection unit 126 are transmitted, via the data communication unit 128, to the evaluation unit 40 located outside a body of the person. The evaluation unit 40 comprises a second processor 420 in two-way communication with a second memory unit 422. The second memory unit 422 comprises a second computer-readable program that causes the second processor 420 to perform the various steps described/explained above when executed on the second processor 420.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Active implantable medical device, comprising a processor, a memory unit, a detection unit configured to detect a cardiac physiologic parameter, and a temperature sensor for sensing a body temperature,
wherein the memory unit comprises a computer-readable program that causes the processor to perform the following steps when executed on the processor:
a) causing the temperature sensor to repeatedly sense a body temperature of a person to whom the active implantable medical device is implanted;
b) storing body temperature values obtained in step a) in the memory unit;
c) causing the detection unit to repeatedly sense a cardiac physiologic parameter of the person;
d) storing cardiac physiologic parameter values obtained in step c) in the memory unit;
e) determining whether at least one of the body temperature values exceeds a predefined body temperature threshold;
f) if an excess of the predefined body temperature threshold has been determined, determining whether at least one of the cardiac physiologic parameter values exceeds a predefined cardiac physiologic parameter threshold, wherein the at least one of the cardiac physiologic parameter values has been determined in the same time period as the at least one of the body temperature values exceeding the body temperature threshold;
g) i) classifying an infection of the person as a local infection if the at least one of the body temperature values exceeds the body temperature threshold and if the at least one of the cardiac physiologic parameter values does not exceed the cardiac physiologic parameter threshold; or ii) classifying an infection of the person as a systemic infection if the at least one of the body temperature values exceeds the body temperature threshold and if the at least one of the cardiac physiologic parameter values exceeds the cardiac physiologic parameter threshold,
wherein the cardiac physiologic parameter is a heart rate or a heart rate at rest, wherein the computer-readable program causes the processor to determine an excess of the cardiac physiologic parameter threshold if the heart rate or the heart rate at rest is at least 110% of a mean heart rate or a mean heart rate at rest, respectively, over a first number of last days prior to the determining, wherein the first number is a number chosen from a range between 3 and 400.

2. Active implantable medical device according to claim 1, wherein the active implantable medical device is an implantable pulse generator, an implantable cardioverter-defibrillator, a cardiac resynchronization therapy device, a neurostimulator, or an implantable cardiac monitor.

3. Active implantable medical device according to claim 1, wherein the cardiac physiologic parameter is at least one chosen from the group consisting of a heart rate, a heart rate at rest, a respiratory rate, a respiratory rate at rest, and a cardiac impedance, cardiac contractility, cardiac output, heart rate variability, respiratory arrhythmia, oxygen saturation, blood flow velocity, heart sounds.

4. Active implantable medical device according to claim 1, wherein the cardiac physiologic parameter is a heart rate or a heart rate at rest, wherein the cardiac physiologic parameter threshold is a heart rate in a range of 80 to 110 beats per minute.

5. Active implantable medical device according to claim 1, wherein the body temperature threshold is a temperature lying in a range from 37.5° C. to 38.5° C.

6. Active implantable medical device according to claim 1, wherein the computer-readable program causes the processor to sense the body temperature multiple times per day.

7. Active implantable medical device according to claim 1, wherein the computer-readable program causes the processor to sense the body temperature for multiple days.

8. Active implantable medical device according to claim 1, wherein the computer-readable program causes the processor to output an alert if the actual body temperature value exceeds the body temperature threshold and if optionally also the cardiac physiologic parameter value exceeds the cardiac physiologic parameter threshold.

9. Arrangement, comprising an active implantable medical device and an evaluation unit separate from the active implantable medical device, wherein the active implantable medical device comprises a first processor, a first memory unit, a detection unit configured to detect a cardiac physiologic parameter, a temperature sensor for sensing a body temperature, and a data communication unit,
wherein the first memory unit comprises a first computer-readable program that causes the first processor to perform the following steps when executed on the first processor:
a) causing the temperature sensor to repeatedly sense a body temperature of a person to whom the active implantable medical device is implanted;
b) causing the detection unit to repeatedly sense a cardiac physiologic parameter of the person;
c) transmitting body temperature values obtained in step a) and cardiac physiologic parameters obtained in step b) via the data communication unit to the evaluation unit located outside a body of the person;
and in that the evaluation unit comprises a second processor and a second memory unit, wherein the second memory unit comprises a second computer-readable program that causes the second processor to perform the following steps when executed on the second processor:
d) storing the transmitted body temperature values and the transmitted cardiac physiologic parameter values in the second memory unit;
e) determining whether at least one of the body temperature values exceeds a predefined body temperature threshold;
f) if an excess of the predefined body temperature threshold has been determined, determining whether at least one of the cardiac physiologic parameter values exceeds a predefined cardiac physiologic parameter threshold, wherein the at least one of the cardiac physiologic parameter values has been determined in the same time period as the at least one of the body temperature values exceeding the body temperature threshold;
g) i) classifying an infection of the person as a local infection if the at least one of the body temperature values exceeds the body temperature threshold and if the at least one of the cardiac physiologic parameter values does not exceed the cardiac physiologic parameter threshold; or ii) classifying an infection of the person as a systemic infection if the at least one of the body temperature values exceeds the body temperature threshold and if the at least one of the cardiac physiologic parameter values exceeds the cardiac physiologic parameter threshold,
wherein the cardiac physiologic parameter is a heart rate or a heart rate at rest, wherein the computer-readable program causes the processor to determine an excess of the cardiac physiologic parameter threshold if the heart rate or the heart rate at rest is at least 110% of a mean heart rate or a mean heart rate at rest, respectively, over a first number of last days prior to the determining, wherein the first number is a number chosen from a range between 3 and 400.

10. Arrangement according to claim 9, wherein the data communication unit is arranged and designed to transfer data to the evaluation unit in a wireless manner.

11. Arrangement according to claim 9, wherein the first computer-readable program causes the first processor to transmit the body temperature values and the cardiac physiologic parameter values at least once a day.

12. Computer program product comprising computer-readable code that causes a processor to perform the following steps when executed on the processor:
a) storing body temperature values obtained from a temperature sensor of an active implantable medical device and cardiac physiologic parameter values obtained from a detection unit of the same active implantable medical device in a memory unit of the implantable medical device or of an evaluation unit located outside a body of a person to whom the active implantable medical device is implanted;
b) determining whether at least one of the body temperature values exceeds a predefined body temperature threshold;
c) if an excess of the predefined body temperature threshold has been determined, determining whether at least one of the cardiac physiologic parameter values exceeds a predefined cardiac physiologic parameter threshold, wherein the at least one of the cardiac physiologic parameter values has been determined in the same time period as the at least one of the body temperature values exceeding the body temperature threshold;
d) i) classifying an infection of the person as a local infection if the at least one of the body temperature values exceeds the body temperature threshold and if the at least one of the cardiac physiologic parameter values does not exceed the cardiac physiologic parameter threshold; or ii) classifying an infection of the person as a systemic infection if the at least one of the body temperature values exceeds the body temperature threshold and if the at least one of the cardiac physiologic parameter values exceeds the cardiac physiologic parameter threshold,
wherein the cardiac physiologic parameter is a heart rate or a heart rate at rest, wherein the computer-readable program causes the processor to determine an excess of the cardiac physiologic parameter threshold if the heart rate or the heart rate at rest is at least 110% of a mean heart rate or a mean heart rate at rest, respectively, over a first number of last days prior to the determining, wherein the first number is a number chosen from a range between 3 and 400.

13. Method for distinguishing between a local infection and a systemic infection of a patient to whom an active implantable medical device has been implanted, the method comprising the following steps:
a) repeatedly sensing a body temperature of a patient to whom an active implantable medical device has been implanted with a temperature sensor of the active implantable medical device, wherein the active implantable medical device further comprises a processor, a memory unit, and a detection unit configured to detect a cardiac physiologic parameter;
b) storing body temperature values obtained in step a) in the memory unit;
c) repeatedly sensing a cardiac physiologic parameter of the patient;
d) storing cardiac physiologic parameter values obtained in step c) in the memory unit;
e) determining whether at least one of the body temperature values exceeds a predefined body temperature threshold;
f) if an excess of the predefined body temperature threshold has been determined, determining whether at least one of the cardiac physiologic parameter values exceeds a predefined cardiac physiologic parameter threshold, wherein the at least one of the cardiac physiologic parameter values has been determined in the same time period as the at least one of the body temperature values exceeding the body temperature threshold;
g) i) classifying an infection of the patient as a local infection if the at least one of the body temperature values exceeds the body temperature threshold and if the at least one of the cardiac physiologic parameter values does not exceed the cardiac physiologic parameter threshold; or ii) classifying an infection of the patient as a systemic infection if the at least one of the body temperature values exceeds the body temperature threshold and if the at least one of the cardiac physiologic parameter values exceeds the cardiac physiologic parameter threshold,
wherein the cardiac physiologic parameter is a heart rate or a heart rate at rest, wherein the computer-readable program causes the processor to determine an excess of the cardiac physiologic parameter threshold if the heart rate or the heart rate at rest is at least 110% of a mean heart rate or a mean heart rate at rest, respectively, over a first number of last days prior to the determining, wherein the first number is a number chosen from a range between 3 and 400.

14. Method according to claim 13, wherein the method further comprises deciding to examine and/or treat the patient in a hospital specialized on the treatment of infectious diseases if the infection of the patient has been classified as systemic infection.

15. Method according to claim 14, wherein the hospital is a hospital specialized on the treatment of COVID-19.
